(19) United States
(12) Patent Application Publication
(10) Pub. No.: US 2009/0175971 A1
(43) Pub. Date: Jul. 9, 2009

(54) METHOD OF USING COMPOSITION COMPRISING POMEGRANATE EXTRACTS AGAINST THE COMMON COLD

(76) Inventor: Mark DREHER, Woodland Hills, CA (US)

Correspondence Address: DALINA LAW GROUP, P.C., 7910 IVANHOE AVE. #325, LA JOLLA, CA, 92037, US

(21) Appl. No.: 11/971,205

(22) Filed: Jan. 8, 2008

(51) Int. Cl. A61K 36/185; A61P 11/00; A61P 31/12

(52) U.S. Cl. 424/769

(57) ABSTRACT

The composition of one or more embodiments of the invention may be used to treat a common cold viral infection, since the composition of the present invention has significant antiviral properties as demonstrated by the examples of this application. The composition of the present invention may also be used as a therapeutic composition to treat one or more symptoms of a viral infection, including, but not limited to, sore throat, congestion, laryngitis, mucositis, and/or mucous membrane inflammation by administration to a subject suffering from one or more of these symptoms or ailments.

### BACKGROUND OF THE INVENTION

1. Field of the Invention

One or more embodiments of the invention relate generally to compositions comprising pomegranate extracts and methods for using thereof as a treatment or preventive against the common cold caused by a number of viruses.

2. Description of the Related Art

It is estimated that there are approximately one billion cases of the common cold in the United States alone, affecting around 1 of every 4 people on a yearly basis. The impact of common cold causing viruses as worldwide pathogens for humans and other mammals is well recognized. According to the Center for Disease Control, 22 million school days are lost annually in the United States due to various strains of the common cold, which is more prevalent in children and younger adults. The common cold has a number of generally well know symptoms such as mucus buildup, difficulty in nasal breathing, sinus swelling, sneezing, a sore throat, cough, and headache. Not all of these symptoms need to be present for the infection to be what is normally considered a common cold. While the common cold is more often caused by one of 100 serotypes of rhinovirus (approximately 30-35% of reported adult colds in the United States), there are a number of other viruses that cause the common cold, including the parainfluenza viruses, coronviruses, adenoviruses and among others. This number of different viruses that can trigger the common cold means that finding a way to treat and protect against all of them is difficult.

As mentioned above, the common cold and its associated symptoms are caused by a wide variety of different viruses. As such there is a need for a composition that provides for one or more anti-viral activities that commonly work against those that cause varieties of the common cold.

### BRIEF SUMMARY OF THE INVENTION

One or more embodiments of the invention are directed to providing a method for reducing the incidence of contracting an illness causing a common cold in mammals.

In the one aspect the invention relates to a method for the prophylactic use of an anti-viral composition to reduce the incidence of contracting an illness. The method comprises the steps of administering to a subject that has been, or will be, exposed to the etiological agents of the common cold, an amount of an anti-viral composition having a composition extracted from fruits of pomegranate; and optionally an acceptable carrier. The amount of anti-viral composition is effective, when administered, to reduce the incidence of contracting the illness.

In another aspect of the invention, a prophylactic anti-viral composition having ingredients obtainable from pomegranate fruit is disclosed. The anti-viral composition is effective, when administered as a nasal spray or as an inhalant spray to a subject that has been, or will be, exposed to the viral etiological agents of the common cold, to reduce the incidence of contracting said illness.

Still another aspect of the inventive method is to provide a method for the treatment or prophylaxis of the common cold, in a subject in need thereof, comprising administering a composition comprising an extract of pomegranate to the skin on or surrounding the nose, including a nasal cavity. Especially provided is a method and composition for controlling the transmission of viruses either by preventing viruses from entering nasal cavities or by effectively controlling viruses introduced into nasal cavities, for example, by contact with a contaminated hand or finger, and continuing to control the viruses for a period of about four or more hours, and up to about 12 hours, after application of the composition to the skin on or surrounding the nose, including a nasal cavity. Administration of the anti-viral composition through injection is also contemplated as being within the scope and spirit of the invention.

These and various other advantages and features of novelty that characterize the invention are pointed out with particularity in the claims annexed hereto and forming a part hereof. However, for a better understanding of the invention, its advantages, and the objects obtained by its use, reference should be made to the accompanying descriptive matter, in which there is described a preferred embodiment of the invention.

### BRIEF DESCRIPTION OF THE DRAWINGS

The above and other aspects, features and advantages of the invention will be more apparent from the following more particular description thereof, presented in conjunction with the following drawings wherein:

FIG. 1 is illustrates inhibition of viral replication by POMEGRANATE JUICE after and during viral absorption into cells.

### DETAILED DESCRIPTION

The composition of the one or more embodiments of the invention may be used to treat the common cold as caused by any number of viral agents, for example but not limited to adenoviruses, rhinoviruses, coronaviruses, any variation of the human parainfluenza virus, since the composition of the present invention has antiviral properties as demonstrated by the examples of this specification. The composition of one or more embodiments of the invention may also be used as a therapeutic composition to treat one or more symptoms of the common cold and other viral infections, including, but not limited to, sore throat, congestion, laryngitis, mucositis, and/or mucous membrane inflammation by administration to a subject suffering from one or more of these symptoms or ailments.

The composition of one or more embodiments of the invention may also be employed to reduce the incidence of contracting an illness. In this application of the invention, a safe and effective amount of the composition of the present invention is administered to a mammal or human that has been or will be exposed to an illness caused by a microbe, to reduce the incidence of contracting said illness, relative to a mammal or human that has been or will be exposed to an illness caused by a microbe to which the composition of the present invention has not been administered.

The composition used in one or more embodiments of the invention may also be formulated with an acceptable carrier. The acceptable carrier may include, but is not limited to: (a) carbohydrates including sweeteners, fructose, sucrose, sugar, dextrose, starch, lactose, maltose, maltodextrins, corn syrup solids, honey solids, commercial tablet nutritional supplements (b) sugar alcohols including mannitol, sorbitol and xylitol; and (c) various relatively insoluble excipients including dicalcium phosphate, calcium sulfate, calcium carbonate, microcrystalline cellulose and other tableting ingredients. Formulated and pursuant to one or more embodiments of the invention, an oral carrier with an effective amount of a composition of the invention can strongly inhibit the viruses on the nasopharynx when administered to a subject in need thereof. Since as outlined above the nasopharynx is an area of high viral breeding, coating the area with a composition of the present invention is preferable to help prevent the viral infection from causing further infections.

The composition of one or more embodiments of the invention may also be formulated into a nasal aerosol or inhalant composition. For these types of formulations, suitable carriers may include the following ingredients: saline with one or more preservatives, absorption promoters to enhance bioavailability, fluorocarbons, and/or conventional solubilizing or dispersion agents.

For purposes of the invention, an extract from pomegranate may be an extract from the whole pomegranate fruit or from any constituents of pomegranate fruit. Examples of constituents of pomegranate fruit that may be used to make the extract of the invention include, but are not limited to, juice, seed, and the inner and outer peel of pomegranate fruit. In one embodiment of the invention, the extract is the juice extract of whole pomegranate fruit. In another embodiment of the invention, the extract is from the inner or outer peel of pomegranate fruit. In a further embodiment of the invention, the extract may be a mixture of two or more extracts of the whole pomegranate or any constituents of pomegranate. The whole fruit of the pomegranate is what is made into an extract. The pomegranate extract may be in powder or liquid form as well as in juice form. The pomegranate extract may also be a mixture that has added in phytochemicals to increase the level above that which is present in the natural pomegranate. Additional anti-oxidants and/or compositions know to have an impact on the common cold such as Zinc may be further mixed in for added to increase effectiveness. While not required pharmaceutical compositions that are for purposes of treating the common cold may be optionally mixed into the composition.

In general, any methods that may produce pomegranate extracts such as juice, powder or a concentrated liquid may be used to provide an effective dose of the composition referenced herein. For the purpose of the invention, the juice may be concentrated or diluted from its natural concentration. The juice may also be mixed with extracts of other constituents of pomegranate to vary the composition. Methods of making the extract, including the juice from whole pomegranate fruits are described in U.S. Pat. No. 6,977,089 entitled “METHODS OF USING POMEGRANATE EXTRACTS FOR CAUSING REGRESSION IN LESIONS DUE TO ARTERIOSCLEROSIS” which is incorporated herein by reference and in U.S. patent application Ser. No. 11/137,248 entitled “PROCESSES FOR EXTRACTING PHYTOCHEMICALS FROM POMEGRANATE SOLIDS AND COMPOSITIONS AND METHODS OF USE THEREOF” which is also incorporated herein by reference.

Extracts from the constituents of pomegranate, i.e., seeds or the inner or outer peel, may be made by various methods. For example, the seeds or the inner or outer peel of pomegranate may be diluted in water and the extract may be made by crushing, squeezing, or extensive vortexing. The insoluble materials of the extract may be separated from the soluble supernatant of the extract. In some instances, the supernatant of the extract is used for the purpose of the invention, although any oily, lipidic fraction of the extract may also be used. The extract from constituents of pomegranate may be concentrated or diluted, or mixed with each other or with pomegranate juice extract.

In accordance with one embodiment of the invention, the extract of the present invention may be prepared by a process including the steps of: (a) crushing and squeezing the whole fruits of the pomegranate, including the inner and outer peels and the seeds, to yield a juice component and an insoluble by-product component, and (b) separating the juice component from the insoluble by-product component. The juice component may be used as a juice extract of the invention. The insoluble by-product component may be resuspended in an aqueous medium, such as, but not limited to, water or alcohol, and be further crushed, squeezed, and mixed to yield a soluble portion and an insoluble portion. Then the soluble portion may be separated from the insoluble portion to produce the extract of the constituents of the invention. Alternatively, the soluble portion may be combined with the juice extract to produce the extract of the invention.

While not specifically required in one embodiment of the invention, the whole fruit of the pomegranate may be enzymatically treated to improve extraction and filtration. For example, pectinase may be used to treat the whole fruit to prevent the formation of pectin gels. Other enzymes may also be used as long as they can improve extraction and filtration of the extract of the invention.

The extract of pomegranate used in accordance with one or more embodiments of the invention may be in a liquid or solid form. In accordance with one embodiment of the invention, a solid form of the extract may be made by lyophilizing the liquid extract of the invention. Alternatively, the constituents of the pomegranate, such as seeds, inner or outer peels, or any insoluble portion discussed above, may be processed directly to form the solid form of the extract of the invention. For example, the constituents of the pomegranate may be dried, and processed into powder or pill forms to be used directly as the solid form of the extract of the invention.

Compositions of one or more embodiments of the invention may be a variety of kinds, including, but not limited to, nutritional supplements, pharmaceutical preparations, vitamin supplements, food additives, or foods supplements. Compositions of the invention may be in convenient dosage forms, including, but not limited to, tablets, suspensions, implants, solutions, emulsions, capsules, powders, syrups, liquid compositions, ointments, lotions, creams, pastes, gels, or the like.

Compositions of one or more embodiments of the invention may include a carrier. Depending on the kind of compositions of the invention, a carrier may be a dietary suitable carrier or a pharmaceutically acceptable carrier, as long as it is compatible with the particular kind of compositions of the invention. Examples of a dietary suitable carrier include, but are not limited to, dietary suitable excipients, diluents, and carriers. Examples of a pharmaceutically acceptable carrier include, but are not limited to, biocompatible vehicles, adjuvants, additives, and diluents to achieve a composition usable as a dosage form. As used herein, the terms “pharmaceutically acceptable,” “physiologically tolerable,” and grammatical variations thereof, as they refer to compositions, carriers, diluents, and reagents, are used interchangeably and represent that the materials are capable of administration to or upon a mammal without the production of undesirable physiological effects.

The compositions of one or more embodiments of the invention may be used alone or in combination with other biologically active ingredients. A composition of embodiments of the invention, alone or in combination with other active ingredients may be administered to a subject in a single dose or multiple doses over a period of time, generally by oral administration. Various administration patterns will be apparent to those skilled in the art. The dosage ranges for the administration of the compositions of the invention are those large enough to produce the desired effect. The dosage should not be so large as to cause any adverse side effects, such as unwanted cross-reactions and the like. Generally, the dosage will vary with the age, weight, sex, condition, and extent of a condition in a subject, and the intended purpose. The dosage can be determined by one of skill in the art without undue experimentation. The dosage can be adjusted in the event of any counter indications, tolerance, or similar conditions. Those of skill in the art can readily evaluate such factors and, based on this information, determine the particular effective concentration of a composition of the invention to be used for an intended purpose.

In one embodiment of the invention, a composition contains the extract of pomegranate in a dosage unit in an amount that contains at least 30 to 10,000 parts per million or 30 to 3,000 mg of polyphenols. For the purpose of the invention, polyphenols are those naturally present in the extract of pomegranate. It should be appreciated that polyphenols are used herein as a measurement marker for the amount of extract used in each dosage unit. Polyphenols are not used herein as being indicative of the only active, ingredients of the extract. It is possible, for example, that other elements of the composition or the synergy of polyphenols and other components of an extract of the invention, are responsible for the activities of the extract.

The term “dosage unit” as used herein refers to physically discrete units suitable as unitary dosages for subjects, each unit containing a predetermined quantity of active material calculated to produce the desired therapeutic effect in association with the required diluent, e.g., a carrier or vehicle. The specifications for the unit dose of this invention are dictated by and are directly dependent on (a) the unique characteristics of the active material and (b) the limitations inherent in the art of compounding such active material for therapeutical use in subjects.

Prophylactic treatment is aimed at a subject that will soon be exposed to the common cold or has recently been exposed to the common cold. Such prophylactic treatment may be effective either alone, or to augment a vaccine or another anti-viral drug. Such prophylactic treatment may also be used against viruses for which there are not yet a vaccine available. In the case of prophylactic treatment, the composition of the invention is administered to a subject that will be exposed to a virus or has recently been exposed to a virus for the purpose of reducing the incidence of active infection by the virus in that subject.

Another aspect of the invention relates to a method of reducing, treating or preventing of at least one symptom or adverse effect of viral infection by administering, to a subject infected with a virus, a composition of the present invention, including ingredients that can be obtained from pomegranate.

In the method, the subject may be a mammal such as a human, an in vitro cell system, or a mammal such as an animal. In the method, the virus that may be inhibited by administration of the pomegranate extract is any virus that cause a symptom of the common cold, which includes, among other viruses, rhinoviruses, influenza viruses, adenovirus, cornavirus, parainfluenza virus, rubella virus, yellow fever virus and respiratory syncytial virus (RSV). Other viruses that can also be inhibited by administration of the pomegranate composition of the present invention includes, among other viruses, West Nile virus, and herpes simplex virus.

The symptoms of the common cold as caused by a viral infection, that may be treated, reduced, or at least partially prevented by this method of the present invention, may include one or more of headache, joint pain, fever, cough, sneezing, muscle ache, running nose, dry mouth, dizziness, and other symptoms related to viral infection.

The effective amount of the composition will vary depending on such factors as the subject being treated, the particular mode of administration, the activity of the particular active ingredients employed, the age, bodyweight, general health, sex and diet of the subject, time of administration, rate of excretion, the particular combination of ingredients employed, the total content of the main ingredient of the composition, and the severity of the illness or symptom. It is within the skill of the person of ordinary skill in the art to account for these factors.

When the composition is administered as a spray, the amounts each of the active ingredients may be reduced as the spray composition delivers the active ingredients more directly to the location where they are needed, as compared to a lozenge or capsule for example.

The following examples are intended to illustrate, but not to limit, the scope of the invention. Indeed, those of ordinary skill in the art can readily envision and produce further embodiments, based on the teachings herein, without undue experimentation. The scope of invention is to be determined by the claims appended hereto.

### EXAMPLE 1

### A Composition of the Present Invention

Pomegranates were hand-picked, washed, chilled to 4° C., and stored in tanks. The fruit was then crushed, squeezed, and treated enzymatically with pectinase to yield the juice and byproducts, which included the inner and outer peels and the seeds. Pectinase hydrolyzes-1,4-galacturonide bonds in pectin, improving extraction and filtration, and prevents formation of pectin gels. Flavonoids constitute 40% (anthocyanins, catechins, and phenols) of total polyphenols in the pomegranate juice. More complex polyphenols are also present in the juice. The pomegranate juice was filtered, pasteurized, concentrated, and stored at −20° C. until use. Pomegranate extracts other than juice are also within the scope and spirit of the invention and as such the invention contemplates use of whole pomegranates in any form such as juice, powder, and concentrated liquid.

### EXAMPLE 2

### Treatment of Sore Throat

The human subjects were found to obtain complete relief from the symptoms of their sore throats after ingesting from 1 to 6 cups of pomegranate juice. It was also found that each cup of pomegranate juice can provide relief from a sore throat for up to 6 hours.

Transmission of cold/flu virus is from person to person usually by personal contact by shaking hands and wipe nose or rub eyes or getting sneeze on and the like.

Mechanism of infection: Upon entry through the nose and the eyes, the virus enters the cells lining the nasopharynx (the area of the upper throat that lies behind the nose) and multiples rapidly. Once viruses reach a high concentration cold/flu symptoms (related to inflammation) rapidly appear.

The nasopharynx is area typically for build high concentration of viruses needed to cause cold or flu symptoms. This build-up of viral concentration is because they are either: (1) trapped by the mucus membranes, which is a natural defense mechanism and/or (2) the viruses concentrate on the outer part of the membranes, where they are less exposed to normal body temperature (since they cannot survive elevated temperatures such as body temperature). The cold/flu viruses are vulnerable on the top of nasopharynx membranes on the upper throat.

Human subjects infected with cold/flu virus not showing symptoms were used as proof of concept. It is desirable keep the virus concentrations low in the upper throat/nasopharynx during the first few days of infection. This prevents the body's defense mechanism, e.g. sneezing, runny nose, fever, malaise and cough, from turning on as the symptoms may confound the study.

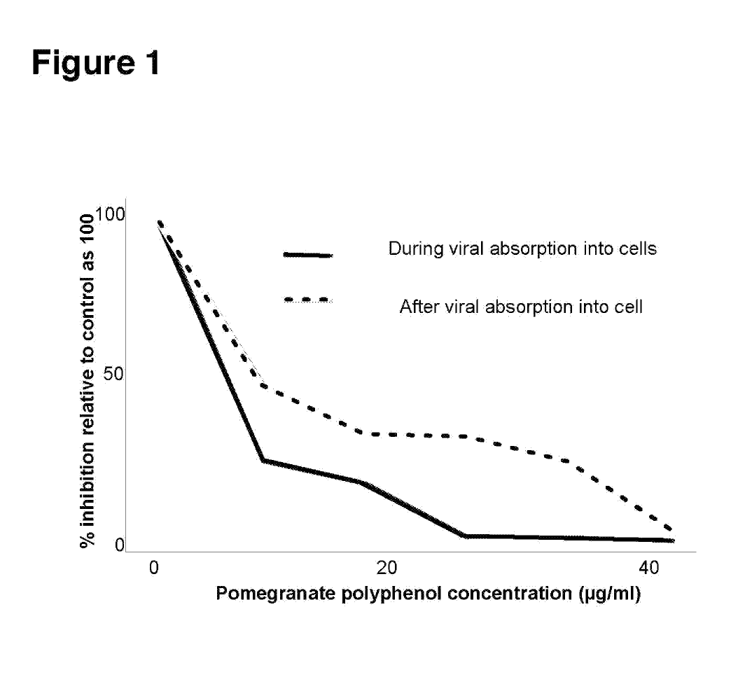

The oral consumption of pomegranate juice strongly inhibits the viruses on the nasopharynx since it coats the lining of the throat/nasopharynx with pomegranate juice. The varying amount of pomegranate juice used to inhibit viral replication on both surface and inner layers of the cells are shown in FIG. 1.

It should be noted that the concentration of polyphenols in pomegranate juice is over 100 times more concentrated than in the diluted pomegranate juice as indicated in the FIG. 1. This high concentrated source of polyphenols in pomegranate juice can immediately inhibit viral replication, protein transport systems, and residual polyphenols are expected to remain for some period in on the cell surface or mucosal layer surrounding the throat/nasopharynx cells. So pomegranate juice is used to treat the nasopharynx which disrupt viral replication and control cold/flu symptom, typically when caught in time before viral levels get too high.

Daily or multi-times per day consumption of pomegranate juice keeps viral concentrations at levels below threshold for symptoms and thus pomegranate extracts may be used as a preventative during the first 1-2 days of exposure.

1. A method of treating a subject infected with a virus that causes symptoms of the common cold comprising:
administering a therapeutically effective amount of a pomegranate extract for at least one day following exposure to a virus that causes symptoms of a common cold, whereby said pomegranate extract further comprises a pomegranate juice from whole pomegranates made by a method comprising the steps of:
washing said whole pomegranates;
chilling said whole pomegranates to about 4° C.;
crushing, squeezing, and treating said whole pomegranates enzymatically with pectinase to yield said pomegranate juice and whole pomegranate byproducts, wherein said pomegranate juice further comprises pectinase hydrolyzes-1,4-galacturonide bonds in pectin, and wherein flavonoids comprise about 40% of total polyphenols in said pomegranate juice.

2-5. (canceled)

6. The method of claim 1 wherein said virus is a rhinovirus.

7. The method of claim 1 wherein said virus is a respiratory syncytial virus (RSV).

8. The method of claim 1 wherein said virus is an adenovirus.

9. The method of claim 1 wherein said virus is a cornavirus.

10. The method of claim 1 wherein said virus is a parainfluenza virus.

11. The method of claim 1 wherein said virus is a rubella virus.

12. The method of claim 1 wherein said virus is a yellow fever virus.

13. The method of claim 1 wherein said subject is a mammal.

14. The method of claim 1 wherein said subject is a human.

15. The method of claim 1 wherein said subject is an in vitro cell.

16. The method of claim 1 wherein said symptoms comprise a sore throat.

17. The method of claim 17 wherein said pomegranate juice further comprises more complex polyphenols.

18. The method of claim 17 further comprising:
filtering, pastureurizing, and concentrating said pomegranate juice; and
storing said pomegranate juice at about −20° C. until said administration.

19. The method of claim 17 wherein said whole pomegranates further comprise the seeds, inner and outer peel of said pomegranates.
